(19) United States
(12) Patent Application Publication
(10) Pub. No.: US 2017/0252521 A1
(43) Pub. Date: Sep. 7, 2017

(54) System For Positioning A Distal End Of A Surgical Device Within A Patient For Surgery And To Administer A Fluid To The Surgery Site

(71) Applicant: Arthrex, Inc., Naples, FL (US)

(72) Inventors: James J. Guerra, Naples, FL (US); Reinhold D. Schmieding, Naples, FL (US); Philip S. O'Quinn, Naples, FL (US)

(73) Assignee: Arthrex, Inc., Naples, FL (US)

(21) Appl. No.: 15/061,687

(22) Filed: Mar. 4, 2016

(51) Int. Cl. A61M 5/32; A61B 34/20; A61M 25/09; A61B 17/16

(52) U.S. Cl. CPC A61M 5/3286; A61B 17/1659; A61B 34/20; A61M 25/09; A61B 2034/2063

(57) ABSTRACT

A system for positioning a distal end of a surgical device within a patient to conduct a surgical procedure or administer a fluid to the patient, or both, is disclosed. The system may use ultrasound to accurately position a surgical device within a patient while being as minimally invasive as possible. As such, the system may include first inserting a needle into a patient under guidance of ultrasound, inserting a guide wire into the needle, removing the needle and inserting a surgical device into the patient along the at least one guide wire. As such, the minimally intrusive needle is first inserted into the patient in a correct position before a larger medical device is inserted to prevent unnecessary intrusion into the patient. In at least one embodiment, the surgical device may be positioned within a patient using a drive handle removably attached to a portion of the surgical device.

### FIELD OF THE DISCLOSURE

The present disclosure generally relates to surgical devices, and more particularly to surgical devices configured to resect tissue and to deliver a biologic healing material to a surgery site.

### BACKGROUND

There exist numerous surgical devices configured to resect tissue from patients via surgical procedures. In some applications, surgical devices may be used to treat recalcitrant golf and tennis elbow. These devices may be inserted into position within a patient by surgeons, and the distal ends of the devices may be moved within patients via the surgeons moving proximal ends of the devices. Accurately positioning the distal ends of the devices has proven challenging.

To promote faster and better healing of surgical sites, technologies have been developed whereby materials are added to the surgical site after a surgical procedure has been conducted. The materials are typically administered to the surgical site as a fluid via one or more tubes inserted into the patient. The materials may work in one or more ways to promote improved healing at the surgical site. Properly administering the materials to a surgical site has proven challenging.

### SUMMARY

A system for positioning a distal end of a surgical device within a patient to conduct a surgical procedure or administer a fluid to the patient, or both, is disclosed. In at least one embodiment, the system may be used to treat recalcitrant golf and tennis elbow or other conditions. The system may be used on human or animal patients. The system may include use of ultrasound to accurately position at least a portion of a surgical device, such as a distal end, within a patient while being as minimally invasive as possible. As such, the system may include first inserting a needle into a patient under guidance of ultrasound, inserting a guide wire into the needle, removing the needle and inserting a surgical device into the patient along the at least one guide wire. The minimally intrusive needle may be first inserted into the patient in a correct position before a larger medical device is inserted to prevent unnecessary intrusion into the patient. In at least one embodiment, the surgical device may be positioned within a patient using a drive handle removably attached to a portion of the surgical device. The drive handle may be removably attached to a drive hub of a distal portion of the surgical device. Once the distal portion of the surgical device has been positioned within a patient, the drive handle may be removed and a proximal portion of the surgical device may be attached to the distal portion to drive the distal portion.

The system may be used via a method of positioning a surgical device within a patient to administer a fluid to the patient including inserting a needle into a patient such that a distal end of the needle is in a desired location within the patient. The method may also include inserting at least one guide wire into the needle positioned at least partially in the patient, such that the at least one guide wire is inserted into the needle a distance whereby a distal end of the at least one guide wire is positioned within the patient. The method may include removing the needle from the patient, leaving the at least one guide wire positioned within the patient. The method may also include inserting at least a portion of a surgical device into the patient along the at least one guide wire. The method may also include attaching a biologic healing source with an inflow port in the surgical device such that a biologic healing fluid may be administered to the patient via the surgical device. The biologic healing fluid may include, but is not limited to, autologous conditioned plasma (ACP), platelet rich plasma (PRP), materials already conceived or discovered or yet to be conceived or discovered.

In another embodiment, the system may be used via a method of positioning a surgical device within a patient to administer a fluid to the patient including inserting a needle with a stylet into a patient while also subjecting a portion of the patient into which the needle is inserted with ultrasound so that a location of a distal end of the needle may be viewed on a graphical user interface to visually determine the location of the needle, thereby enabling the distal end of the needle to be positioned in the desired location within the patient. The method may also include withdrawing the stylet from the needle after the needle has been inserted into the patient. The method may include inserting one or more guide wires into the needle positioned at least partially in the patient, such that the guide wire is inserted into the needle a distance whereby a distal end of the guide wire is positioned within the patient. The method may also include removing the needle from the patient, leaving the guide wire positioned within the patient. The method may include positioning the guide wire within a guide wire receiving channel The method may also include inserting at least a portion of a surgical device into the patient along the guide wire such that the guide wire guides the surgical device into position.

The system may also include a positioning system for a surgical device including a drive handle body including a handle configured to be grasped by a user, such as a surgeon. The system may include a first drive coupled to the drive handle body and configured to mate with a drive hub of a distal portion of the surgical device, and a second drive coupled to the drive handle body and configured to mate with a drive hub of a distal portion of another surgical device. The first and second drives may be sized differently. The first drive may be positioned at a first end of the drive handle body, and the second drive may be positioned at a second end of the drive handle body. The second drive at the second end of the drive handle may be positioned on an opposite end of the drive handle from the first drive at the first end. The drive handle is versatile in that the drive handle may be used together with different sized drive hubs on the distal portions of surgical devices, such as, but not limited to, rotary shavers.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a side view of a drive handle with a distal portion of a surgical device attached to a first drive on a first end of the drive handle and a distal portion of another surgical device attached to a second drive on a second end of the drive handle, whereby the first and second drives may be different sizes.

FIG. 2 is a partial cross-sectional view of a needle with stylet inserted into a patient and into contact with an epicondyle of the patient.

FIG. 3 is a partial cross-sectional view of a needle inserted into a patient and into contact with an epicondyle of the patient and the stylet being removed from the needle.

FIG. 4 is a partial cross-sectional view of a needle inserted into a patient and into contact with an epicondyle of the patient and a guide wire being positioned to be inserted into the needle.

FIG. 5 is a partial cross-sectional view of a needle inserted into a patient and into contact with an epicondyle of the patient and a guide wire inserted into the needle.

FIG. 6 is a partial cross-sectional view of a guide wire positioned in the patient after the needle has been removed.

FIG. 7 is a side view of the guide wire in the patient and a small incision made at the intersection of the guide wire and the outer surface of the skin to enable a distal portion of a surgical device to be inserted into the patient.

FIG. 8 is a partial cross-sectional view of a guide wire in the patient and a distal portion of a surgical device inserted into the patient by placing the guide wire in a guide wire receiving channel to guide a distal end of the surgical device into proper position within the patient, and includes a detail view of a rotary shaver head attached to a distal end of the distal portion of the surgical device.

FIG. 9 is a partial cross-sectional view of a distal portion of a surgical device positioned in the patient and the guide wire removed from the guide wire receiving channel on the distal portion of a surgical device.

FIG. 10 is a partial cross-sectional view of a distal portion of a surgical device positioned in the patient and the drive handle disconnected from the distal portion of the surgical device.

FIG. 11 is a partial cross-sectional view of a distal portion of a surgical device positioned in the patient, a proximal end of the surgical device attached to the distal portion of the surgical device, a fluid source attached to an inflow port on the guide wire receiving channel, and a biologic healing source.

FIG. 12 is a flow chart of the method of positioning a surgical device within a patient to conduct surgery or administer a fluid to the patient using the system, or both.

FIG. 13 is a perspective view of a graphical user interface displaying results of an ultrasound image of a needle positioned at a surgical site within the patient.

FIG. 14 is a perspective view of a guide wire positioned in an animal patient, such as, but not limited to, a horse, and a surgeon guiding a distal portion of a surgical device into the patient by placing the guide wire in a guide wire receiving channel to guide a distal end of the surgical device into proper position within the patient.

FIG. 15 is a perspective view of a surgeon grasping a proximal portion of a surgical device to control movement of a distal end of the distal portion of the surgical device in the patient.

FIG. 16 is a perspective view of a surgeon grasping a proximal portion of a surgical device to control movement of a distal end of the distal portion of the surgical device in the patient and an ultrasound wand in contact with the patient's skin to show the surgeon the location of the distal end of the distal portion of the surgical device in the patient.

### DETAILED DESCRIPTION

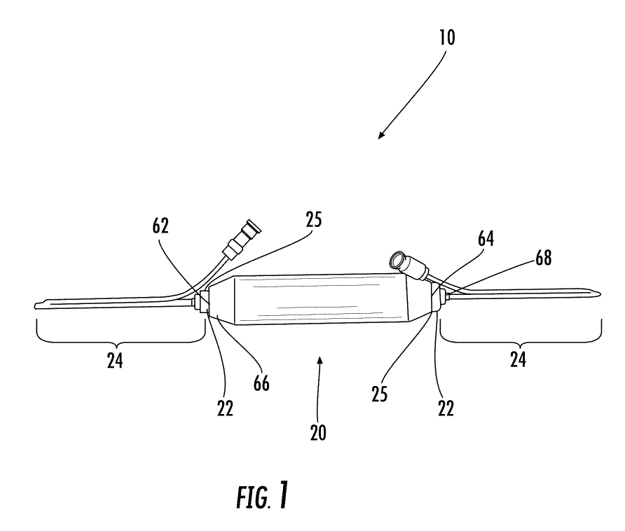

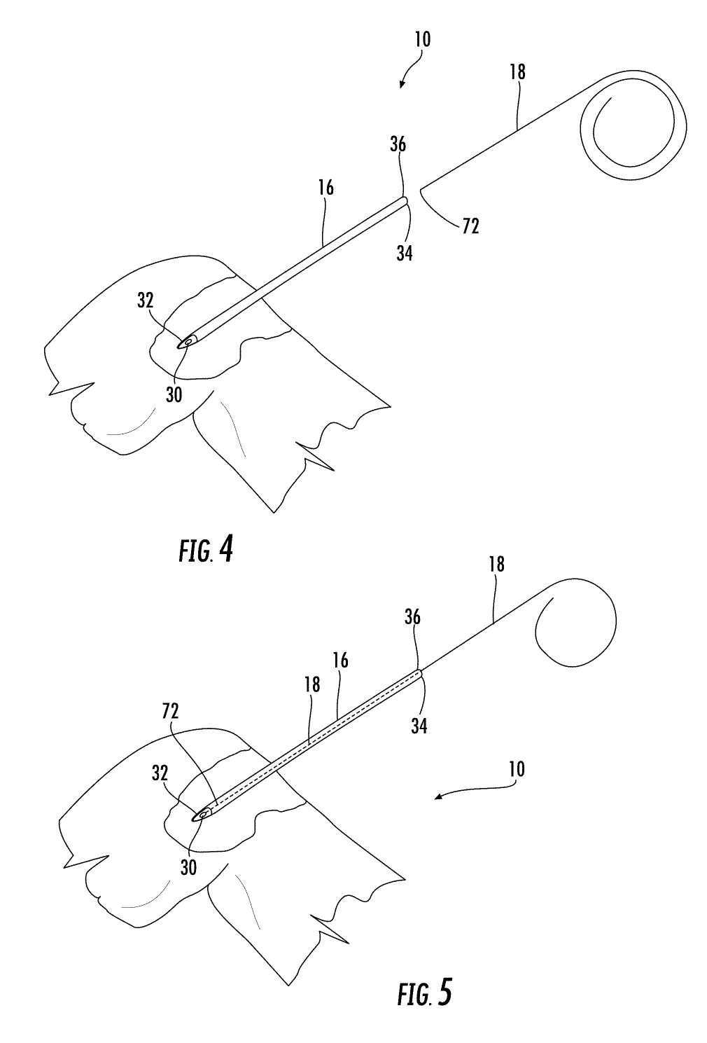

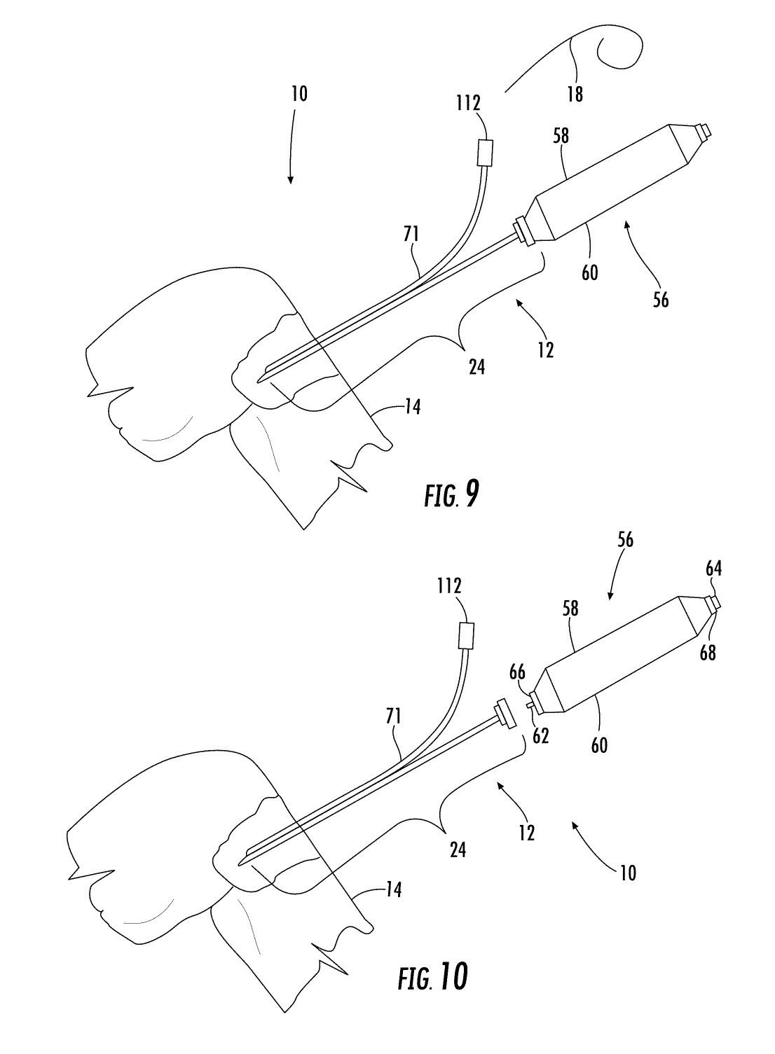

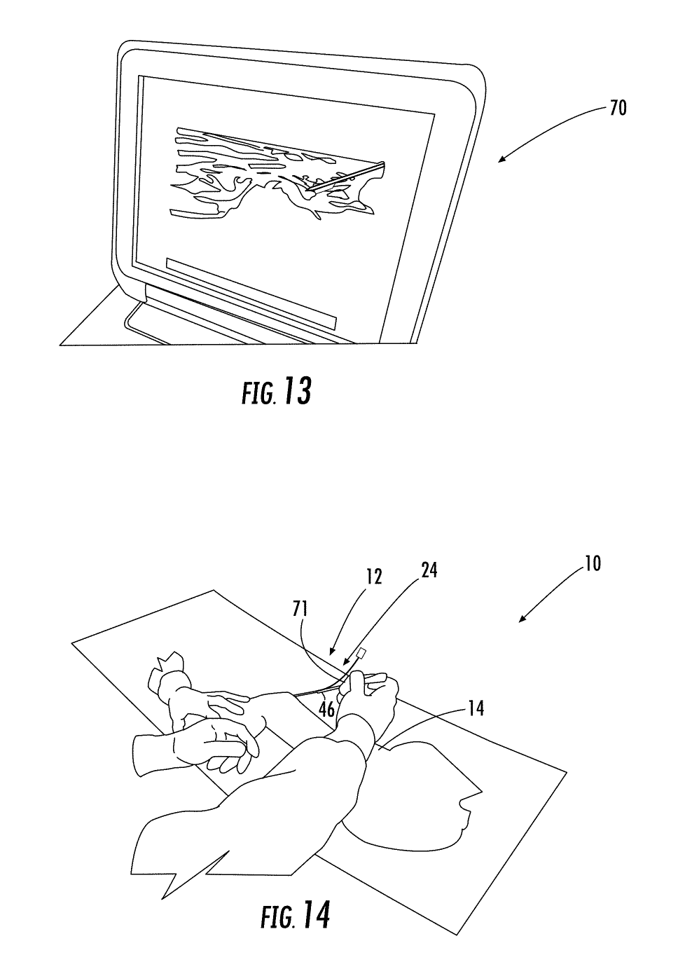

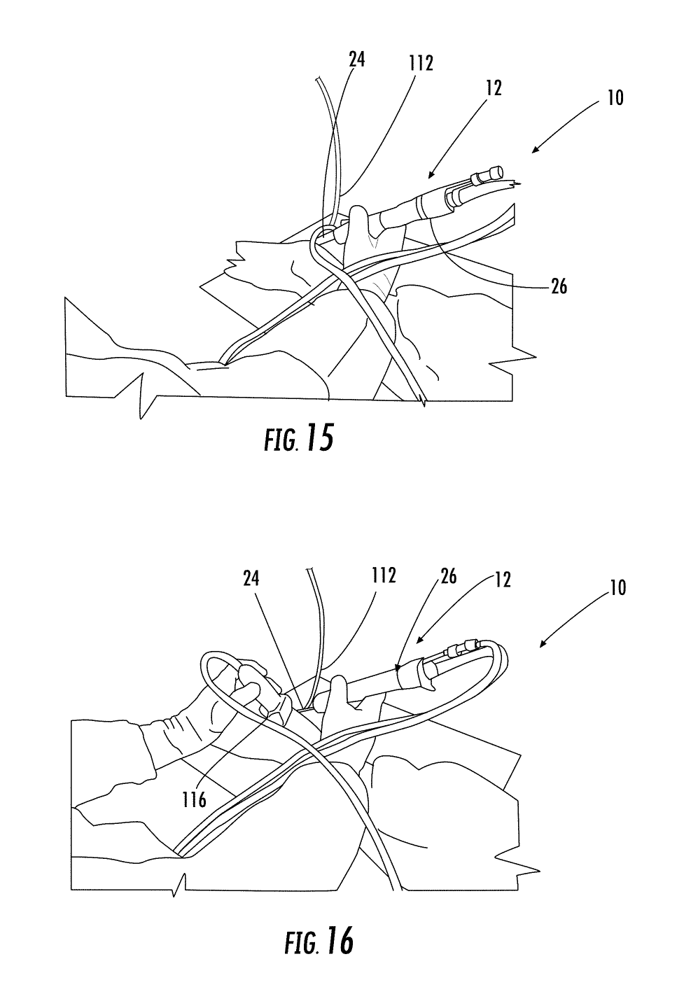

As shown in FIGS. 1-16, a system 10 for positioning a distal end 45 of a surgical device 12 within a patient 14 to conduct a surgical procedure or administer a fluid to the patient, or both, is disclosed. In at least one embodiment, the system 10 may be used to treat recalcitrant golf and tennis elbow and other conditions. The system 10 may be used on human or animal patients. The system 10 may include use of ultrasound to accurately position at least a portion of a surgical device 12 within a patient 14 while being as minimally invasive as possible. As such, the system 10 may include first inserting a needle 16 into a patient 14 under guidance of ultrasound, inserting a guide wire 18 into the needle 16, removing the needle 16 and inserting a surgical device 12 into the patient 14 along the guide wire 18. As such, the minimally intrusive needle 16 is first inserted into the patient 14 in a correct position before a larger medical device 12 is inserted to prevent unnecessary intrusion into the patient 14. In at least one embodiment, the surgical device 12 may be positioned within a patient 14 using a drive handle 20 removably attached to a portion of the surgical device 12. The drive handle 20 may be removably attached to a drive hub 22 of a distal portion 24 of the surgical device 12. Once the distal portion 24 of the surgical device 12 has been positioned within a patient 14, the drive handle 20 may be removed and a proximal portion 26, as shown in FIGS. 11, 15 and 16, of the surgical device 12 may be attached to the distal portion 24 to drive the distal portion 24.

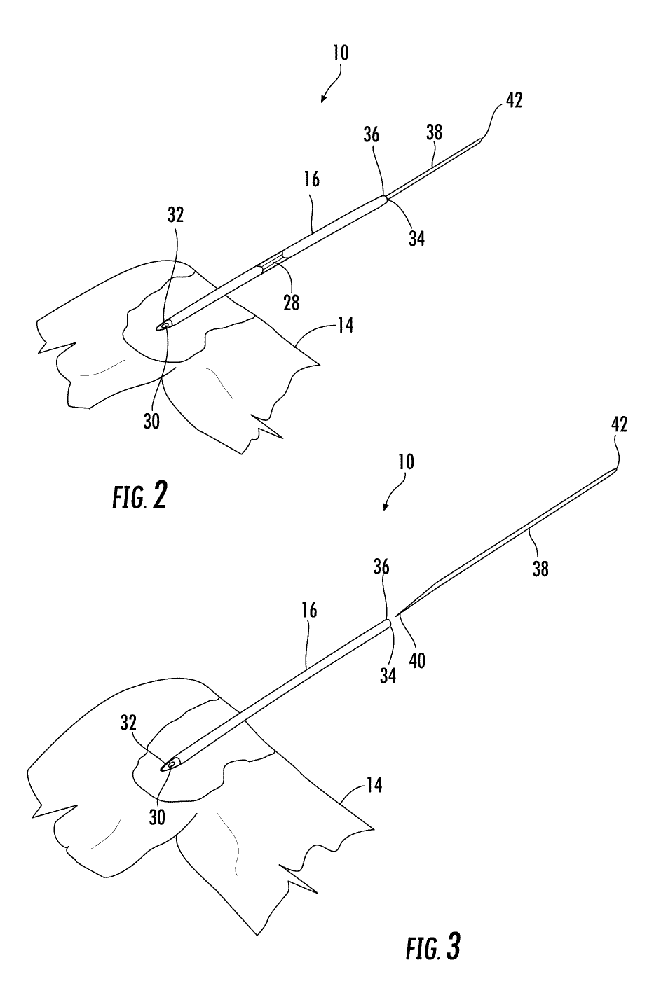

The system 10 may include a needle 16, as shown in FIGS. 2 and 3, that facilitates insertion of a guide wire 18 into a patient. The needle 16 may have sufficient rigidity to resist bending while being forced into tissue. The needle 16 may be formed from any appropriate material, such as, but not limited to stainless steel. In at least one embodiment, the needle 16 may include a hollow internal channel 28 extending throughout the needle 16. The internal channel 28 may include a first opening 30 at a distal end 32 and a second opening 34 at a proximal end 36. The internal channel 28 may have any appropriate size and shape. In at least one embodiment, the needle 16 may be, but is not limited to being, an 18 gauge spinal needle. In at least one embodiment, a stylet 38 may be positioned within the needle 16, as shown in FIG. 2. The stylet 38 may be sized such that the a distal end 40 of the stylet 38, as shown in FIG. 3, may be positioned at the first opening 30 at the distal end 32 while a proximal end 42 of the stylet 38 extends proximally from the second opening 34 at the proximal end 36 of the needle 16. With such a configuration, the stylet 38 may be withdrawn from the internal channel 28 of the needle 16, as shown in FIG. 3, by simply grasping the stylet 38 and withdrawing the stylet 38 from the needle 16. In at least one embodiment, the stylet 38 may be formed from a cylindrical shaft having an outer diameter less than a diameter of the internal channel 28 of the needle 16. The stylet 38 may be formed from any appropriate material, such as, but not limited to stainless steel.

The system 10 may include a guide wire 18 usable to be inserted into the internal channel 28 of the needle 16. The guide wire 18 may have sufficient mechanical properties causing the guide wire 18 to exhibit sufficient stiffness such that the guide wire 18 may be inserted into the internal channel 28 of the needle 16 at the second opening 34 at the proximal end 36 and may be pushed to the distal end 32. In at least one embodiment, the guide wire 18 may be formed from a flexible nitinol wire.

The system 10 may include a surgical device 12 formed from a distal portion 24 and a proximal portion 26, as shown in FIGS. 11, 15 and 16. The distal portion 24 of the surgical device 12 may be formed from any appropriate medical device. In at least one embodiment, as shown in FIG. 8, the distal portion 24 may be formed from a rotary shaver 44 formed from a rotary shaver housing 46 with a rotary shaver drive shaft 48 contained therein. A rotary shaver head 50 may extend from a distal end 45 of the rotary shaver drive shaft 48 and may be configured to resect tissue via rotary motion. The distal portion 24 may include a drive hub 22 attached to a proximal end 25 of the distal portion 24. The drive hub 22 may be configured to form an interference fit with the first or second drives 62, 64 of the drive handle 20. The distal portion 24 may also include a guide wire receiving channel 71, as shown in FIGS. 8-11, configured to receive the guide wire 18 to guide the distal portion 24 into a patient 14. In at least one embodiment, the guide wire receiving channel 71 may be a cylindrical guide tube. The guide wire receiving channel 71 may have other cross-sectional configurations other than circular, as in the tube configuration. The guide wire receiving channel 71 may also support the inflow port 112. As such, the inflow port 112 may provide fluids or biologic fluids to a surgical site via the guide wire receiving channel 71.

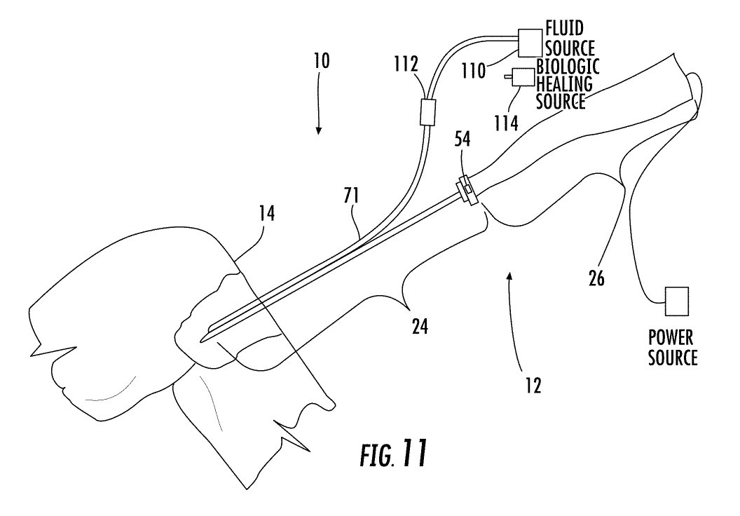

The proximal portion 26 of the surgical device 12 may have a drive 54 configured to mate with the drive hub 22, as shown in FIG. 11. The drive 54 may be in mechanical communication with a motor configured to impart rotary motion to the drive 54. The proximal portion 26 may be removably coupled to the distal portion 24. In at least one embodiment, the distal portion 24 of the surgical device 12 may be inserted into a patient with the proximal portion 26 attached to the surgical device 12. In another embodiment, the distal portion 24 of the surgical device 12 may be inserted into a patient with a positioning system 56, as shown in FIGS. 1 and 8-10. In at least one embodiment, the positioning system 56 may be formed from a drive handle 20 attached to the distal portion 24 via a drive 54 that mates with the drive hub 22 attached to the proximal end of the distal portion 24. The drive handle 20 may be configured to enable a surgeon to insert the distal portion 24 of the surgical device 12 into a patient 14. The drive handle 20 may include a drive handle body 58 including a handle 60 configured to be grasped by a user, such as, but not limited to, a surgeon. The handle 60 may have any appropriate configuration. In at least one embodiment, as shown in FIG. 10, the drive handle 20 may include a first drive 62 coupled to the drive handle body 58 and configured to mate with the drive hub 22 of the distal portion 24 of the surgical device 12. The drive handle 20 may also include a second drive 64 coupled to the drive handle body 58 and configured to mate with a drive hub of a distal portion of another surgical device 12. The second drive 64 may be sized differently than the first drive 62. In at least one embodiment, the second drive 64 may be sized larger than the first drive 62. The first and second drives 62, 64 may be sized differently to accommodate different sizes in drive hubs 22 offered by a single manufacturer or by different manufacturers. The first and second drives 62, 64 having different sizes enable the drive handle 12 to be attached to rotary shavers and other rotary devices having different sized drive hubs.

In at least one embodiment, as shown in FIGS. 1 and 10, the first drive 62 may be positioned at a first end 66 of the drive handle body 58, and the second drive 64 may be positioned at a second end 68 of the drive handle body 58. The second drive 64 at the second end 68 of the drive handle body 58 may be positioned on an opposite end of the drive handle body 58 from the first drive 62 at the first end 66. When in use, either end of the drive handle 12 may be used by simply changing the orientation of the drive handle 12 to use the first or second drive 62, 64. The drive handle body 58 may be configured to be grasped by a surgeon and may be positioned between the first and second drives 62, 64 at the first and second ends 66, 68.

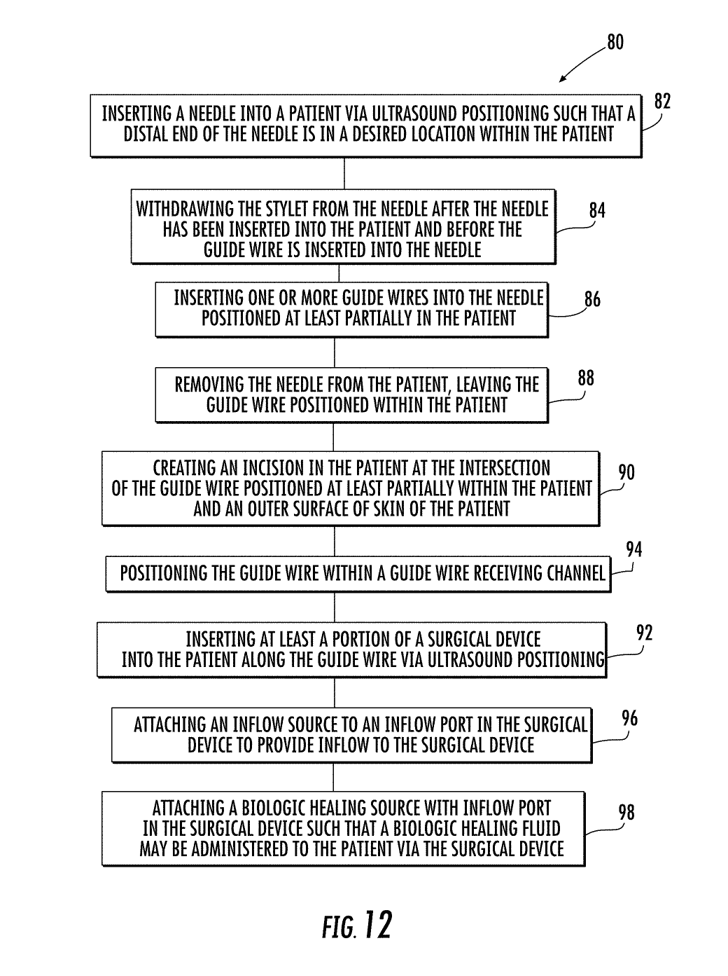

As shown schematically in FIG. 12, a method 80 of positioning a surgical device 12 within a patient 14 to conduct surgery or administer a fluid to the patient using the system 10, or both, may include inserting a needle 16 at 82 into a patient 14 such that a distal end 32 of the needle 16 is in a desired location within the patient 14, as shown in FIG. 2. In at least one embodiment, the step of inserting the needle 16 at 82 into a patient 14 such that the distal end 32 of the needle 16 is in a desired location within the patient 14 includes inserting the needle 16 with ultrasound, such as with an ultrasound wand 116, as shown in FIG. 16, so that the location of the needle 16 may be viewed on a graphical user interface 70, as shown in FIG. 14, to visually determine the location of the needle 16, thereby enabling the distal end 32 of the needle 16 to be positioned in the desired location within the patient 14. In at least one embodiment, the desired location is at an epicondyle of the patient 14. The step of inserting a needle 16 at 82 into a patient 14 may include inserting a needle 16 into a patient 14 whereby the needle 16 includes a stylet 38 positioned within a hollow internal chamber 28 in the needle 16, as shown in FIG. 2. The method 80 may also include withdrawing the stylet 38 at 84 from the needle 16 after the needle 16 has been inserted into the patient 14 and before the guide wire 18 is inserted into the needle 16, as shown in FIG. 3. The stylet 38 may be removed by a user grabbing a portion of the stylet 38 extending proximally from the needle 16 and withdrawing the stylet 38 from the needle 16.

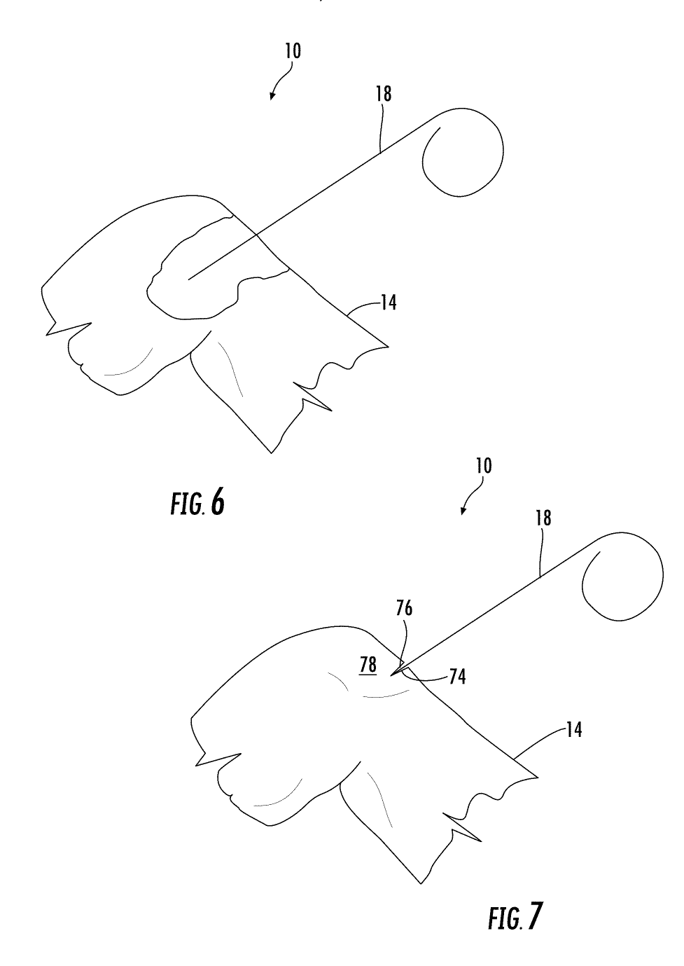

The method 80 may include inserting one or more guide wires 18 at 86 into the needle 16 positioned at least partially in the patient 14 such that the guide wire 18 is inserted into the needle 16 a distance such that a distal end 72 of the guide wire 18 is positioned within the patient 14, as shown in FIG. 5. In at least one embodiment, the guide wire 18 may be inserted such that a distal end 72 of the guide wire 18 is positioned at the first opening 30 of the hollow internal channel 28 at the distal end 32 of the needle 16. The method 80 may include removing the needle 16 at 88 from the patient 14, which leaves the guide wire 18 positioned within the patient 14, as shown in FIG. 6.

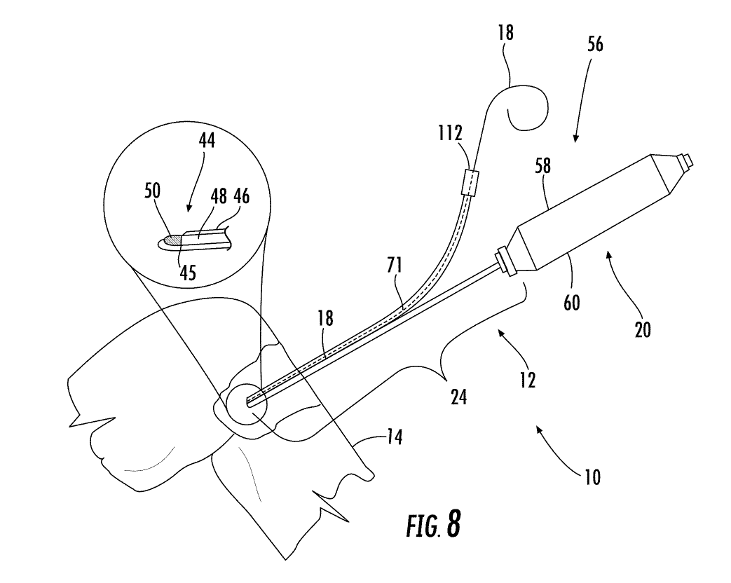

The method 80 may include creating an incision 74, as shown in FIG. 7, at 90 in the patient 14 at an intersection 76 of the guide wire 18 positioned at least partially within the patient 14 and an outer surface 78 of skin of the patient 14, whereby the incision 74 may be sized to receive the portion of the surgical device 12 inserted into the patient 14 along the guide wire 18. The method 80 may include inserting at least a portion of a surgical device 12 at 90 into the patient 14 along the guide wire 18, as shown in FIG. 8. The method 80 may include positioning the guide wire 18 within a guide wire receiving channel 71 at 94 and inserting at least a portion of a surgical device 12 into the patient 14 along the guide wire 18. The step of inserting at least a portion of a surgical device 12 at 92 into the patient 14 along the guide wire 18 may include inserting a distal end 32 of a rotary shaver 44 along the guide wire 18. The handle 60 may be used to insert the cylindrical rotary shaver 44 containing the rotary shaver head 50 and the cylindrical guide wire receiving channel 71 configured to receive the guide wire 18 into the patient 14.

The step of inserting at least a portion of a surgical device 12 at 92 into the patient 14 along the guide wire 18 may include inserting a distal portion 24 of a rotary shaver 44 along the guide wire 18, wherein the rotary shaver head 50 of the rotary shaver 44 is placed into contact with an epicondyle of the patient 14. The step of inserting at least a portion of a surgical device 12 at 92 into the patient 14 along the guide wire 18 may include inserting at least a portion of a distal portion 24 of a two piece surgical device 12 formed from the distal portion 24 and a proximal portion 26, wherein the distal portion 24 may be formed from a rotary shaver housing 46 with a rotary shaver head 50 contained therein, a guide wire receiving channel 71 may be coupled to the rotary shaver housing 46, and a drive hub 22 may be attached to a proximal end 25 of the distal portion 24. The step of inserting at least a portion of a surgical device 12 at 92 into the patient 14 along the guide wire 18 may include inserting at least a portion of the distal portion 24 of the two piece surgical device 12 with a drive handle 20 attached to the distal portion 24 via at least one drive 54 that mates with the drive hub 22 attached to a proximal end 25 of the distal portion 24. Further, the step of inserting at least a portion of a surgical device 12 at 92 into the patient 14 along the guide wire 18 may include inserting at least a portion of the distal portion 24 of the two piece surgical device 12 with a drive handle 20 having a first drive 62 that is sized differently than a second drive 64 on the drive handle 20, whereby the first drive 62 is attached to the distal portion 24 via matting attachment to the drive hub 22 attached to a proximal end 25 of the distal portion 24. Once inserted into a patient 14, the handle 60 may be disengaged from the drive hub 22, and the rotary shaver drive of the proximal portion 26 of the surgical device 12 may be attached to the drive hub 22 to drive the rotary shaver head 50 within the patient 14, as shown in FIG. 10. Once the distal portion 24 of the surgical device 12 has been positioned within a patient, a proximal portion 26 of the surgical device 12 may be attached to the distal portion 24 by mating the drive 54 with the drive hub 22, as shown in FIG. 11.

The method 80 may include attaching an inflow source 110 at 96 to an inflow port 112 in the surgical device 12 to provide inflow to the surgical device 12, as shown in FIG. 11. The inflow fluid may be any fluid, such as, but not limited to, any fluid usable to enable the surgical procedure, to enhance healing and the like. The inflow port 112 may be coupled to the guide wire receiving channel 71, as shown in FIGS. 8-11. The method 80 may include guiding a distal end 45 of the surgical device 12 during use of the surgical device 12 via input received from an ultrasound of the portion of the patient 14 into which the needle 16 is inserted, thereby enabling surgeon controlling a distal end 45 of the surgical device 12 to view a location of a distal end 45 of the surgical device 12 via viewing a graphical user interface 70, as shown in FIG. 14, displaying ultrasound results of the portion of the patient 14 into which the needle 16 is inserted. During operation of the surgical device 12, the surgical device 12, such as, but not limited to being, a rotary shaver 22, may have a small cutting diameter and operate in an oscillating spinning motion to resect bone or tissue, or both, from an operating site, which may be within a patient. A user, such as, but not limited to being, a surgeon, may easily position the rotary shaver head 50 because the user is able to sense great feeling with the device 12 as the rotary shaver head 50 at the distal end 45 of the rotary shaver drive shaft 48 is moved along the epicondyle of the patient 14. As such, the user via feeling feedback from the device 12, is able to confirm proper placement tactilely as well as visually with the ultrasound and graphical user interface 70, as shown in FIG. 14, displaying the ultrasound image of the distal end 45 of the rotary shaver drive shaft 48 positioned within the patient 14.

The method 80 may also include attaching a biologic healing source 114 at 98 with the inflow port 112 in the surgical device 12 such that a biologic healing fluid may be administered to the patient 14 via the surgical device 12. The biologic healing fluid may include, but is not limited to, autologous conditioned plasma (ACP), platelet rich plasma (PRP), materials already conceived or discovered or yet to be conceived or discovered. The inflow port 112 may be configured to deliver fluid to the distal end 45 of the rotary shaver drive shaft 48 at the rotary shaver head 50.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A method of positioning a surgical device within a patient to administer a fluid to the patient, comprising:
inserting a needle into a patient such that a distal end of the needle is in a desired location within the patient;
inserting at least one guide wire into the needle positioned at least partially in the patient, such that the at least one guide wire is inserted into the needle a distance whereby a distal end of the at least one guide wire is positioned within the patient;
removing the needle from the patient, leaving the at least one guide wire positioned within the patient; and
inserting at least a portion of a surgical device into the patient along the at least one guide wire.

2. The method of claim 1, wherein inserting the needle into a patient such that the distal end of the needle is in a desired location within the patient comprises inserting a needle into a patient while also subjecting the portion of the patient into which the needle is inserted with ultrasound so that the location of the needle may be viewed on a graphical user interface to visually determine the location of the needle, thereby enabling the distal end of the needle to be positioned in the desired location within the patient.

3. The method of claim 1, wherein inserting a needle into a patient such that a distal end of the needle is in a desired location within the patient comprises inserting a needle into a patient whereby the needle includes a stylet positioned within a hollow chamber in the needle and further comprising withdrawing the stylet from the needle after the needle has been inserted into the patient and before the at least one guide wire is inserted into the needle.

4. The method of claim 1, wherein inserting at least a portion of the surgical device into the patient along the at least one guide wire further comprises positioning the at least one guide wire within a guide wire receiving channel and inserting at least a portion of a surgical device into the patient along the at least one guide wire.

5. The method of claim 1, further comprises creating an incision in the patient at an intersection of the at least one guide wire positioned at least partially within the patient and an outer surface of skin of the patient, whereby the incision is sized to receive the portion of the surgical device inserted into the patient along the at least one guide wire.

6. The method of claim 1, wherein inserting at least a portion of a surgical device into the patient along the at least one guide wire comprises inserting a distal portion of a rotary shaver along the at least one guide wire.

7. The method of claim 1, wherein inserting at least a portion of a surgical device into the patient along the at least one guide wire comprises inserting a distal portion of a rotary shaver along the at least one guide wire, wherein a rotary shaver head of the rotary shaver is placed into contact with an epicondyle of the patient.

8. The method of claim 1, wherein inserting at least a portion of a surgical device into the patient along the at least one guide wire comprises inserting at least a portion of a distal portion of a two piece surgical device formed from the distal portion and a proximal portion, wherein the distal portion is formed from a rotary shaver housing with a rotary shaver head contained therein, a guide wire receiving channel coupled to the rotary shaver housing, and a drive hub attached to a proximal end of the distal portion.

9. The method of claim 8, wherein inserting at least a portion of a surgical device into the patient along the at least one guide wire comprises inserting at least a portion of the distal portion of the two piece surgical device with a drive handle attached to the distal portion via at least one drive that mates with the drive hub attached to a proximal end of the distal portion.

10. The method of claim 8, wherein inserting at least a portion of a surgical device into the patient along the at least one guide wire comprises inserting at least a portion of the distal portion of the two piece surgical device with a drive handle having a first drive that is sized differently than a second drive on the drive handle, whereby the first drive is attached to the distal portion via matting attachment to the drive hub attached to a proximal end of the distal portion.

11. The method of claim 1, further comprising attaching an inflow source to an inflow port in the surgical device to provide inflow to the surgical device.

12. The method of claim 1, further comprising guiding a distal end of the surgical device during use of the surgical device via input received from an ultrasound of the portion of the patient into which the needle is inserted, thereby enabling surgeon controlling a distal end of the surgical device to view a location of a distal end of the surgical device via viewing a graphical user interface displaying ultrasound results of the portion of the patient into which the needle is inserted.

13. The method of claim 1, further comprising attaching a biologic healing source with an inflow port in the surgical device such that a biologic healing fluid may be administered to the patient via the surgical device.

14. A method of positioning a surgical device within a patient to administer a fluid to the patient, comprising:
inserting a needle with a stylet into a patient while also subjecting a portion of the patient into which the needle is inserted with ultrasound so that a location of a distal end of the needle may be viewed on a graphical user interface to visually determine the location of the needle, thereby enabling the distal end of the needle to be positioned in the desired location within the patient;
withdrawing the stylet from the needle after the needle has been inserted into the patient;
inserting at least one guide wire into the needle positioned at least partially in the patient, such that the at least one guide wire is inserted into the needle a distance whereby a distal end of the at least one guide wire is positioned within the patient;
removing the needle from the patient, leaving the at least one guide wire positioned within the patient;
positioning the at least one guide wire within a guide wire receiving channel; and
inserting at least a portion of a surgical device into the patient along the at least one guide wire.

15. The method of claim 14, wherein inserting at least a portion of a surgical device into the patient along the at least one guide wire comprises inserting at least a portion of a distal portion of a two piece surgical device formed from the distal portion and a proximal portion, wherein the distal portion is formed from a rotary shaver housing with a rotary shaver head contained therein, a guide wire receiving channel coupled to the rotary shaver housing, and a drive hub attached to a proximal end of the distal portion.

16. The method of claim 15, wherein inserting at least a portion of a surgical device into the patient along the at least one guide wire comprises inserting at least a portion of the distal portion of the two piece surgical device with a drive handle having a first drive that is sized differently than a second drive on the drive handle, whereby the first drive is attached to the distal portion via matting attachment to the drive hub attached to a proximal end of the distal portion.

17. The method of claim 14, further comprising guiding a distal end of the surgical device during use of the surgical device via input received from an ultrasound of the portion of the patient into which the needle is inserted, thereby enabling surgeon controlling a distal end of the surgical device to view a location of a distal end of the surgical device via viewing a graphical user interface displaying ultrasound results of the portion of the patient into which the needle is inserted.

18. The method of claim 14, further comprising attaching a biologic healing source with an inflow port in the surgical device such that a biologic healing fluid may be administered to the patient via the surgical device.

19. A positioning system for a surgical device, comprising:
a drive handle body including a handle configured to be grasped by a user;
a first drive coupled to the drive handle body and configured to mate with a drive hub of a distal portion of the surgical device; and
a second drive coupled to the drive handle body and configured to mate with a drive hub of a distal portion of another surgical device, wherein the second drive is sized differently than the first drive.

20. The positioning system of claim 19, wherein the first drive is positioned at a first end of the drive handle bod, and the second drive is positioned at a second end of the drive handle body, wherein the second drive at the second end of the drive handle is positioned on an opposite end of the drive handle from the first drive at the first end.
